SEQUENCE LISTING (73 sequences)

SEQ ID NO: 1 - DNA, 597 bp, Artificial Sequence
  Chemically synthesized, SARS-CoV-2_RBD
aatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 2 - protein, 197 aa, Artificial Sequence
  Chemically synthesized, SARS-CoV-2_RBD
NITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGP

SEQ ID NO: 3 - DNA, 597 bp, Artificial Sequence
  Chemically synthesized, SARS-CoV-2_ RBD_2
aacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggaccctaatga

SEQ ID NO: 4 - protein, 197 aa, Artificial Sequence
  Chemically synthesized, SARS-CoV-2_ RBD_2
NITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGP

SEQ ID NO: 5 - DNA, 597 bp, Artificial Sequence
  Chemically synthesized, SARS-CoV-2_ RBD_3
aatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 6 - protein, 197 aa, Artificial Sequence
  Chemically synthesized, SARS-CoV-2_ RBD_3
NITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGP

SEQ ID NO: 7 - DNA, 597 bp, Artificial Sequence
  Chemically synthesized, SARS-CoV-2_ RBD_4
aacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaatga

SEQ ID NO: 8 - protein, 197 aa, Artificial Sequence
  Chemically synthesized, SARS-CoV-2_ RBD_4
NITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 9 - DNA, 1320 bp, Artificial Sequence
  Chemically synthesized, SARS-CoV-2_ RBD dimer
cgagtccaacccactgagagcatcgtaagatttccgaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagtctacaaacctcgtaaagaataagcgggtacagccgaccgaatcaatagtccgattccccaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggcctaagaagagcactaatttggtgaagaacaaatagtga

SEQ ID NO: 10 - protein, 438 aa, Artificial Sequence
  Chemically synthesized, SARS-CoV-2_ RBD dimer
RVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNK

SEQ ID NO: 11 - DNA, 1032 bp, Artificial Sequence
  Chemically synthesized, 180
ctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaa

SEQ ID NO: 12 - protein, 344 aa, Artificial Sequence
  Chemically synthesized, 180
LSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLE

SEQ ID NO: 13 - DNA, 498 bp, Artificial Sequence
  Chemically synthesized, FR
ctgtccaaggacataatcaaactgctcaacgaacaggtgaacaaggagatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagc

SEQ ID NO: 14 - protein, 166 aa, Artificial Sequence
  Chemically synthesized, FR
LSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 15 - DNA, 210 bp, Artificial Sequence
  Chemically synthesized, IMX
aaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtg

SEQ ID NO: 16 - protein, 70 aa, Artificial Sequence
  Chemically synthesized, IMX
KKQGDADVCGEVAYIQSVVSDCHVPTAELRTLLEIRKLFLEIQKLKVELQGLSKELRFGIVASRANHALV

SEQ ID NO: 17 - DNA, 210 bp, Artificial Sequence
  Chemically synthesized, IMX_2
aagaaacaaggcgacgccgatgtgtgtggggaagttgcctacattcaatctgtcgtcagcgattgtcatgtccccacagccgaattgcgaacccttctcgaaattaggaagctctttcttgaaatccaaaagctcaaggtggaattgcaaggactcagtaaagagcttagatttggcatagtagcgtccagagccaaccatgcccttgtc

SEQ ID NO: 18 - protein, 70 aa, Artificial Sequence; identical to SEQ ID NO: 16
  Chemically synthesized, IMX_2

SEQ ID NO: 19 - DNA, 462 bp, Artificial Sequence
  Chemically synthesized, LS
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcga

SEQ ID NO: 20 - protein, 154 aa, Artificial Sequence
  Chemically synthesized, LS
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 21 - DNA, 1815 bp, Artificial Sequence
  Chemically synthesized, LS3_RBD_gJohn_180_pVAX
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 22 - protein, 603 aa, Artificial Sequence
  Chemically synthesized, LS3_RBD_gJohn_180_pVAX
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGP

SEQ ID NO: 23 - DNA, 1815 bp, Artificial Sequence
  Chemically synthesized, LS3_RBD_gKylie_180_pVAX
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggaccctaatga

SEQ ID NO: 24 - protein, 603 aa, Artificial Sequence
  Chemically synthesized, LS3_RBD_gKylie_180_pVAX
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGP

SEQ ID NO: 25 - DNA, 1815 bp, Artificial Sequence
  Chemically synthesized, LS3_RBD_gDan_180_pVAX
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 26 - protein, 603 aa, Artificial Sequence
  Chemically synthesized, LS3_RBD_gDan_180_pVAX
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGP

SEQ ID NO: 27 - DNA, 1815 bp, Artificial Sequence
  Chemically synthesized, LS3_RBD_gPenta_180_pVAX
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaatga

SEQ ID NO: 28 - protein, 603 aa, Artificial Sequence
  Chemically synthesized, LS3_RBD_gPenta_180_pVAX
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 29 - DNA, 1254 bp, Artificial Sequence
  Chemically synthesized, LS3_RBD_gJohn_FR_pVAX
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtctgtggacctggtggcagtggcggctctggtggttcaggcggcagtggtggtgggctgtccaaggacataatcaaactgctcaacgaacaggtgaacaaggagatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 30 - protein, 416 aa, Artificial Sequence
  Chemically synthesized, LS3_RBD_gJohn_FR_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 31 - DNA, 1254 bp, Artificial Sequence
  Chemically synthesized, LS3_RBD_gKylie_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 32 - protein, 416 aa, Artificial Sequence
  Chemically synthesized, LS3_RBD_gKylie_FR_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 33 - DNA, 1254 bp, Artificial Sequence
  Chemically synthesized, LS3_RBD_gDan_FR_pVAX
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctacagtatgtggtcctggcggctccggtggctctggcgggagtggcggatcaggtggtggactgagtaaggacatcatcaaacttctcaacgaacaggtgaacaaggagatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 34 - protein, 416 aa, Artificial Sequence
  Chemically synthesized, LS3_RBD_gDan_FR_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 35 - DNA, 1254 bp, Artificial Sequence
  Chemically synthesized, LS3_RBD_gPenta_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 36 - protein, 416 aa, Artificial Sequence
  Chemically synthesized, LS3_RBD_gPenta_FR_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 37 - DNA, 966 bp, Artificial Sequence
  Chemically synthesized, LS3_RBD_gJohn_IMX_pVAX
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggcccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 38 - protein, 320 aa, Artificial Sequence
  Chemically synthesized, LS3_RBD_gJohn_IMX_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 39 - DNA, 966 bp, Artificial Sequence
  Chemically synthesized, LS3_RBD_gKylie_IMX_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 40 - protein, 320 aa, Artificial Sequence
  Chemically synthesized, LS3_RBD_gKylie_IMX_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 41 - DNA, 966 bp, Artificial Sequence
  Chemically synthesized, LS3_RBD_gDan_IMX_pVAX
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggcccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 42 - protein, 320 aa, Artificial Sequence
  Chemically synthesized, LS3_RBD_gDan_IMX_pVAX
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 43 - DNA, 966 bp, Artificial Sequence
  Chemically synthesized, LS3_RBD_gPenta_IMX_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 44 - protein, 320 aa, Artificial Sequence
  Chemically synthesized, LS3_RBD_gPenta_IMX_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 45 - DNA, 981 bp, Artificial Sequence
  Chemically synthesized, LS3_RBD_gPenta_IMX_6His_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgcaccaccatcatcactaatag

SEQ ID NO: 46 - protein, 325 aa, Artificial Sequence
  Chemically synthesized, LS3_RBD_gPenta_IMX_6His_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALVHHHHH

SEQ ID NO: 47 - DNA, 1629 bp, Artificial Sequence
  Chemically synthesized, RBD_gPenta_dimer_IMX_pVAX
atggattggacgtggatattgttcctggtagccgctgcgactcgggtccatagccgagtccaacccactgagagcatcgtaagatttccgaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagtctacaaacctcgtaaagaataagcgggtacagccgaccgaatcaatagtccgattccccaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggcctaagaagagcactaatttggtgaagaacaaaggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtagtga

SEQ ID NO: 48 - protein, 541 aa, Artificial Sequence
  Chemically synthesized, RBD_gPenta_dimer_IMX_pVAX
MDWTWILFLVAAATRVHSRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKGGSGGSGGSGGSGGGKKQGDADVCGEVAYIQSVVSDCHVPTAELRTLLEIRKLFLEIQKLKVELQGLSKELRFGIVASRANHALV

SEQ ID NO: 49 - DNA, 1602 bp, Artificial Sequence
  Chemically synthesized, RBDg5_IMX_RBDg5_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcggttccggtggatcaggtgggagcggtggatcaggggggggtaagaaacaaggcgacgccgatgtgtgtggggaagttgcctacattcaatctgtcgtcagcgattgtcatgtccccacagccgaattgcgaacccttctcgaaattaggaagctctttcttgaaatccaaaagctcaaggtggaattgcaaggactcagtaaagagcttagatttggcatagtagcgtccagagccaaccatgcccttgtcggtggcagtggcggttccggtggttccggtggcagtggcggtggtaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggccttaatag

SEQ ID NO: 50 - protein, 532 aa, Artificial Sequence
  Chemically synthesized, RBDg5_IMX_RBDg5_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALVGGSGGSGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 51 - DNA, 1158 bp, Artificial Sequence
  Chemically synthesized, RBD_gJohn_LS_pVAX
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatag

SEQ ID NO: 52 - protein, 384 aa, Artificial Sequence
  Chemically synthesized, RBD_gJohn_LS_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGP

SEQ ID NO: 53 - DNA, 1158 bp, Artificial Sequence
  Chemically synthesized, RBD_gKylie_LS_pVAX
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggaccctaatag

SEQ ID NO: 54 - protein, 384 aa, Artificial Sequence
  Chemically synthesized, RBD_gKylie_LS_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGP

SEQ ID NO: 55 - DNA, 1158 bp, Artificial Sequence
  Chemically synthesized, RBD_gDan_LS_pVAX
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatag

SEQ ID NO: 56 - protein, 384 aa, Artificial Sequence
  Chemically synthesized, RBD_gDan_LS_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGP

SEQ ID NO: 57 - DNA, 1158 bp, Artificial Sequence
  Chemically synthesized, RBD_gPenta_LS_pVAX
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaatag

SEQ ID NO: 58 - protein, 384 aa, Artificial Sequence
  Chemically synthesized, RBD_gPenta_LS_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 59 - DNA, 1140 bp, Artificial Sequence
  Chemically synthesized, RBD_gPenta_LS_short_pVAX
atggattggacgtggatattgttcctggtagccgctgcgactcgggtccatagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggagggagtggaggctcaggaggaggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctagtga

SEQ ID NO: 60 - protein, 378 aa, Artificial Sequence
  Chemically synthesized, RBD_gPenta_LS_short_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 61 - DNA, 1644 bp, Artificial Sequence
  Chemically synthesized, NTD_gTri4_LS3_RBD_gPenta_pVAX
atggattggacctggattctgtttctcgtggcagccgcaacaagagtgcatagccaatgcgttaatttgacgaccaggacacagctcccgcccgcatataccaacagtttcactaggggagtttactacccagataaagtgttccggtcttccgtattgcacagcacccaggacctgttccttcccttcttctctaacgtgacgtggtttcatgccatccatgtctcaggaaccaacggcaccaagagattcgacaacccggttttgccttttaatgacggtgtatactttgccagcactgaaaagtcaaatattattaggggttggatatttggcaccacgctggattcaaagactcaaagcctgcttatcgtcaataatgcaacgaatgtcgttatcaaggtctgcgagttccagttctgcaacgatagctttctgggcgtatactaccataagaacaacaagagctggatggagagcgaattccgggtttactccagtgctaataactgcactttcgaatacgtatcacaaccgttcctcatggatctcgaaggcaaacagggcaacttcaagaatcttcgggaatttgtgtttaagaatattgatggttactttaagatatactctaaacataccccaatcaacctcgttagggacctgccccaagggttttcagccctggagcctctcgtggacctgccaatcggtatcaacatcactcgctttcagacgttgctcgctcttcacaggtcttacttgacgaacgggagctccagctcagggtggactgccggcgcagcagcatattatgtcggatatctccagccgcgaacatttctgctcaaatataatgaaaacggaacaattaccgatgcagtagactgtgccctcgaccccgctagcgaaacaaagtgcaccaacaaaagctttacagccgaagggagcggttcaggtagtggatctggtagtcttagatttggcatagttgcatcccgggcaaaccatgccctcgttggatctgggagtgggtcaggaagcgggtccaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaataa

SEQ ID NO: 62 - protein, 546 aa, Artificial Sequence
  Chemically synthesized, NTD_gTri4_LS3_RBD_gPenta_pVAX
MDWTWILFLVAAATRVHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDSFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTNGSSSSGWTAGAAAYYVGYLQPRTFLLKYNENGTITDAVDCALDPASETKCTNKSFTAEGSGSGSGSGSLRFGIVASRANHALVGSGSGSGSGSNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 63 - DNA, 1644 bp, Artificial Sequence
  Chemically synthesized, NTD_LS3_RBD_gPenta_pVAX
atggattggacctggattctgtttctcgtggcagccgcaacaagagtgcatagccaatgcgttaatttgacgaccaggacacagctcccgcccgcatataccaacagtttcactaggggagtttactacccagataaagtgttccggtcttccgtattgcacagcacccaggacctgttccttcccttcttctctaacgtgacgtggtttcatgccatccatgtctcaggaaccaacggcaccaagagattcgacaacccggttttgccttttaatgacggtgtatactttgccagcactgaaaagtcaaatattattaggggttggatatttggcaccacgctggattcaaagactcaaagcctgcttatcgtcaataatgcaacgaatgtcgttatcaaggtctgcgagttccagttctgcaacgatccgtttctgggcgtatactaccataagaacaacaagagctggatggagagcgaattccgggtttactccagtgctaataactgcactttcgaatacgtatcacaaccgttcctcatggatctcgaaggcaaacagggcaacttcaagaatcttcgggaatttgtgtttaagaatattgatggttactttaagatatactctaaacataccccaatcaacctcgttagggacctgccccaagggttttcagccctggagcctctcgtggacctgccaatcggtatcaacatcactcgctttcagacgttgctcgctcttcacaggtcttacttgacgcctggggattccagctcagggtggactgccggcgcagcagcatattatgtcggatatctccagccgcgaacatttctgctcaaatataatgaaaacggaacaattaccgatgcagtagactgtgccctcgaccccgctagcgaaacaaagtgcaccgccaaaagctttacagccgaaggtagtgggagcggatcagggagcggaagtttgcggtttggtattgtggcctcacgggcaaaccatgccctcgtcggtagcgggtcaggaagcggaagcgggtcaaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaataa

SEQ ID NO: 64 - protein, 546 aa, Artificial Sequence
  Chemically synthesized, NTD_LS3_RBD_gPenta_pVAX
MDWTWILFLVAAATRVHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPASETKCTAKSFTAEGSGSGSGSGSLRFGIVASRANHALVGSGSGSGSGSNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 65 - DNA, 1899 bp, Artificial Sequence
  Chemically synthesized, NTD_gTri4_LS3_RBD_g5_IMX_pVAX
atggattggacctggattctgtttctcgtggcagccgcaacaagagtgcatagccaatgcgttaatttgacgaccaggacacagctcccgcccgcatataccaacagtttcactaggggagtttactacccagataaagtgttccggtcttccgtattgcacagcacccaggacctgttccttcccttcttctctaacgtgacgtggtttcatgccatccatgtctcaggaaccaacggcaccaagagattcgacaacccggttttgccttttaatgacggtgtatactttgccagcactgaaaagtcaaatattattaggggttggatatttggcaccacgctggattcaaagactcaaagcctgcttatcgtcaataatgcaacgaatgtcgttatcaaggtctgcgagttccagttctgcaacgatagctttctgggcgtatactaccataagaacaacaagagctggatggagagcgaattccgggtttactccagtgctaataactgcactttcgaatacgtatcacaaccgttcctcatggatctcgaaggcaaacagggcaacttcaagaatcttcgggaatttgtgtttaagaatattgatggttactttaagatatactctaaacataccccaatcaacctcgttagggacctgccccaagggttttcagccctggagcctctcgtggacctgccaatcggtatcaacatcactcgctttcagacgttgctcgctcttcacaggtcttacttgacgaacgggagctccagctcagggtggactgccggcgcagcagcatattatgtcggatatctccagccgcgaacatttctgctcaaatataatgaaaacggaacaattaccgatgcagtagactgtgccctcgaccccgctagcgaaacaaagtgcaccaacaaaagctttacagccgaaggaagtggaagcggatcaggtagtggtagcctcagatttggcatagtggcctcacgagccaaccatgcactcgtcggaagcggatcaggaagcgggtctggtagcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaataa

SEQ ID NO: 66 - protein, 631 aa, Artificial Sequence
  Chemically synthesized, NTD_gTri4_LS3_RBD_g5_IMX_pVAX
MDWTWILFLVAAATRVHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDSFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTNGSSSSGWTAGAAAYYVGYLQPRTFLLKYNENGTITDAVDCALDPASETKCTNKSFTAEGSGSGSGSGSLRFGIVASRANHALVGSGSGSGSGSNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 67 - DNA, 1881 bp, Artificial Sequence
  Chemically synthesized, RBD_g5_dimer_LS_pVAX
atggattggacgtggatattgttcctggtagccgctgcgactcgggtccatagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggccgagtccaacccactgagagcatcgtaagatttccgaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagtctacaaacctcgtaaagaataagcgggtacagccgaccgaatcaatagtccgattccccaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggcctaagaagagcactaatttggtgaagaacaaatagtga

SEQ ID NO: 68 - protein, 625 aa, Artificial Sequence
  Chemically synthesized, RBD_g5_dimer_LS_pVAX
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNK

SEQ ID NO: 69 - protein, 18 aa, Artificial Sequence
  Chemically synthesized, IgE leader sequence
MDWTWILFLVAAATRVHS

SEQ ID NO: 70 - DNA, 45 bp, Artificial Sequence
  Chemically synthesized, linker
ggaggctccggaggatctggagggagtggaggctcaggaggaggc

SEQ ID NO: 71 - protein, 15 aa, Artificial Sequence
  Chemically synthesized, linker
GGSGGSGGSGGSGGG

SEQ ID NO: 72 - DNA, 60 bp, Artificial Sequence
  Chemically synthesized, LS3 linker
ttgcgatttggtattgtcgcttcccgcgcaaaccatgcgctcgtgggtggttccggtggc

SEQ ID NO: 73 - protein, 20 aa, Artificial Sequence
  Chemically synthesized, LS3 linker
LRFGIVASRANHALVGGSGG

(19) United States
(12) Patent Application Publication
(10) Pub. No.: US 2024/0009300 A1
(43) Pub. Date: Jan. 11, 2024

(54) DNA ENCODED NANOPARTICLES AND METHOD OF USE THEREOF AS A CORONAVIRUS DISEASE 2019 (COVID-19) VACCINE

(71) Applicant: The Wistar Institute of Anatomy and Biology, Philadelphia, PA (US)

(72) Inventors: Daniel Kulp, Moorestown, NJ (US); Ziyang Xu, Philadelphia, PA (US); David Weiner, Merion (PA)

(21) Appl. No.: 18/251,620

(22) PCT Filed: Nov. 3, 2021
(86) PCT No.: PCT/US21/57859

§ 371 Date: May 3, 2023

Related U.S. Application Data
(60) Provisional application No. 63/109,123, filed on Nov. 3, 2020.

(51) Int. Cl. A61K 39/215; A61P 31/14; C07K 14/165

(52) U.S. Cl. CPC A61K 39/215; A61P 31/14; C07K 14/165

(57) ABSTRACT

Disclosed herein are nanoparticles comprising one or more SARS coronavirus 2 (SARS-CoV-2) Spike receptor binding domain (RBD) antigen and nucleic acid molecules encoding the same. Also disclosed herein is a method of treating a SARS-COV-2 infection or treating or preventing a disease or disorder associated therewith in a subject in need thereof, by administering the nanoparticles, or encoding nucleic acid molecules, to the subject.

### CROSS REFERENCE TO RELATED APPLICATIONS

This application claims priority to U.S. Provisional Application No. 63/109,123, filed Nov. 3, 2020, which is hereby incorporated by reference herein in its entirety.

### BACKGROUND

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, OC43, NL63, and HKU1 are endemic in the human population.

COVID-19, known previously as 2019-nCoV pneumonia or disease, has rapidly emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. In December 2019 the city of Wuhan in China became the epicenter for an outbreak of the novel coronavirus, SARS-CoV-2. SARS-CoV-2 was isolated and sequenced from human airway epithelial cells from infected patients (Zhu et al., 2020 N Engl J Med, 382:727-733; Wu et al., 2020, Nature, 579:265-269). Disease symptoms can range from mild flu-like to severe cases with life-threatening pneumonia (Huang et al., 2020, Lancet, 395:497-506). The global situation is dynamically evolving, and on Jan. 30, 2020 the World Health Organization declared COVID-19 as a public health emergency of international concern (PHEIC) and on Mar. 11, 2020 it was declared a global pandemic. As of Apr. 1, 2020 there are 932,605 people infected and 46,809 deaths (gisaid.org/epiflu-applications/global-cases-covid-19). Infections have spread to multiple continents. Human-to-human transmission has been observed in multiple countries, and a shortage of disposal personal protective equipment, and prolonged survival times of coronaviruses on inanimate surfaces (Hulkower et al., 2011, Am J Infect Control 39, 401-407), have compounded this already delicate situation and heightened the risk of nosocomial infections. Advanced research activities must be pursued in parallel to push forward protective modalities in an effort to protect billions of vulnerable individuals worldwide. Currently, no licensed preventative vaccine or specific anti-viral therapy is available for COVID-19.

Accordingly, a need remains in the art for the development of a safe and effective vaccine for the treatment of SARS-CoV-2 infection or the treatment or prevention of a disease or disorder associated with SARS-CoV-2 infection such as COVID-19.

### SUMMARY OF THE INVENTION

In one embodiment, the invention relates to an immunogenic composition comprising a nucleic acid molecule encoding a SARS-CoV-2 spike protein receptor binding domain (RBD). In one embodiment, the nucleotide sequence encodes a peptide comprising an amino acid sequence having at least about 90% identity over an entire length of an amino acid sequence of SEQ ID NO:2, SEQ ID NO:4, SEQ ID NO:6 or SEQ ID NO:8. In one embodiment, the nucleotide sequence encodes SEQ ID NO:2, SEQ ID NO:4, SEQ ID NO:6 or SEQ ID NO:8. In one embodiment, the nucleotide sequence encodes at least two peptides, wherein each of the at least two peptides comprises an amino acid sequence having at least about 90% identity over an entire length of an amino acid sequence of SEQ ID NO:2, SEQ ID NO:4, SEQ ID NO:6 or SEQ ID NO:8. In one embodiment, the nucleotide sequence encodes at least two of SEQ ID NO:2, SEQ ID NO:4, SEQ ID NO:6 and SEQ ID NO:8.

In one embodiment, the nucleotide sequence comprises at least about 90% identity over an entire length of the nucleic acid sequence of SEQ ID NO:1, SEQ ID NO:3, SEQ ID NO:5 or SEQ ID NO:7. In one embodiment, the nucleotide sequence comprises SEQ ID NO:1, SEQ ID NO:3, SEQ ID NO:5 or SEQ ID NO:7. In one embodiment, the nucleotide sequence encodes at least two peptides, wherein each of the encoding sequences comprises at least about 90% identity over an entire length of a nucleotide sequence of SEQ ID NO:1, SEQ ID NO:3, SEQ ID NO:5 or SEQ ID NO:7. In one embodiment, the nucleotide sequence comprises at least two of SEQ ID NO:1, SEQ ID NO:3, SEQ ID NO:5 and SEQ ID NO:7.

In one embodiment, the nucleic acid molecule further encodes an oligomerization domain. In one embodiment, the nucleic acid molecule encodes a peptide comprising an amino acid sequence having at least about 90% identity over an entire length of an amino acid sequence of SEQ ID NO:12, SEQ ID NO:14, SEQ ID NO:16, SEQ ID NO:18 or SEQ ID NO:20. In one embodiment, the nucleic acid molecule encodes a peptide comprising SEQ ID NO:12, SEQ ID NO:14, SEQ ID NO:16, SEQ ID NO:18 or SEQ ID NO:20. In one embodiment, the nucleic acid molecule comprises a nucleotide sequence having at least about 90% identity over an entire length of a nucleotide sequence of SEQ ID NO:11, SEQ ID NO:13, SEQ ID NO:15, SEQ ID NO:17 or SEQ ID NO:19. In one embodiment, the nucleic acid molecule comprises a nucleotide sequence of SEQ ID NO:11, SEQ ID NO:13, SEQ ID NO:15, SEQ ID NO:17 or SEQ ID NO:19.

In one embodiment, the nucleic acid molecule encodes a self-assembling nanoparticle comprising an amino acid sequence having at least about 90% identity over an entire length of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the nucleic acid molecule encodes a self-assembling nanoparticle comprising an amino acid sequence of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the nucleic acid molecule encodes a self-assembling nanoparticle comprising a fragment comprising at least 60% of the full length amino acid sequence of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68.

In one embodiment, the nucleic acid molecule encoding the self-assembling nanoparticle comprises a nucleotide sequence having at least about 90% identity over an entire length of a nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. In one embodiment, the nucleic acid molecule encoding the self-assembling nanoparticle comprises a nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. In one embodiment, the nucleic acid molecule encoding the self-assembling nanoparticle comprises a fragment comprising at least 60% of the full length nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67.

In one embodiment, the nucleic acid molecule comprises an expression vector.

In one embodiment, the nucleic acid molecule is incorporated into a viral particle.

In one embodiment, the immunogenic composition further comprises a pharmaceutically acceptable excipient.

In one embodiment, the immunogenic composition further comprises an adjuvant.

In one embodiment, the invention relates to a nucleic acid molecule encoding a SARS-CoV-2 spike protein receptor binding domain (RBD). In one embodiment, the nucleotide sequence encodes a peptide comprising an amino acid sequence having at least about 90% identity over an entire length of an amino acid sequence of SEQ ID NO:2, SEQ ID NO:4, SEQ ID NO:6 or SEQ ID NO:8. In one embodiment, the nucleotide sequence encodes SEQ ID NO:2, SEQ ID NO:4, SEQ ID NO:6 or SEQ ID NO:8. In one embodiment, the nucleotide sequence encodes at least two peptides, wherein each of the at least two peptides comprises an amino acid sequence having at least about 90% identity over an entire length of an amino acid sequence of SEQ ID NO:2, SEQ ID NO:4, SEQ ID NO:6 or SEQ ID NO: 8. In one embodiment, the nucleotide sequence encodes at least two of SEQ ID NO:2, SEQ ID NO:4, SEQ ID NO:6 and SEQ ID NO:8.

In one embodiment, the nucleotide sequence comprises at least about 90% identity over an entire length of the nucleic acid sequence of SEQ ID NO:1, SEQ ID NO:3, SEQ ID NO:5 or SEQ ID NO:7. In one embodiment, the nucleotide sequence comprises SEQ ID NO:1, SEQ ID NO:3, SEQ ID NO:5 or SEQ ID NO:7. In one embodiment, the nucleotide sequence encodes at least two peptides, wherein each of the encoding sequences comprises at least about 90% identity over an entire length of a nucleotide sequence of SEQ ID NO:1, SEQ ID NO:3, SEQ ID NO:5 or SEQ ID NO:7. In one embodiment, the nucleotide sequence comprises at least two of SEQ ID NO:1, SEQ ID NO:3, SEQ ID NO:5 and SEQ ID NO:7.

In one embodiment, the nucleic acid molecule further encodes an oligomerization domain. In one embodiment, the nucleic acid molecule encodes a peptide comprising an amino acid sequence having at least about 90% identity over an entire length of an amino acid sequence of SEQ ID NO:12, SEQ ID NO:14, SEQ ID NO:16, SEQ ID NO:18 or SEQ ID NO:20. In one embodiment, the nucleic acid molecule encodes a peptide comprising SEQ ID NO:12, SEQ ID NO:14, SEQ ID NO:16, SEQ ID NO:18 or SEQ ID NO:20. In one embodiment, the nucleic acid molecule comprises a nucleotide sequence having at least about 90% identity over an entire length of a nucleotide sequence of SEQ ID NO:11, SEQ ID NO:13, SEQ ID NO:15, SEQ ID NO:17 or SEQ ID NO:19. In one embodiment, the nucleic acid molecule comprises a nucleotide sequence of SEQ ID NO:11, SEQ ID NO:13, SEQ ID NO:15, SEQ ID NO:17 or SEQ ID NO:19.

In one embodiment, the nucleic acid molecule encodes a self-assembling nanoparticle comprising an amino acid sequence having at least about 90% identity over an entire length of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the nucleic acid molecule encodes a self-assembling nanoparticle comprising an amino acid sequence of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the nucleic acid molecule encodes a self-assembling nanoparticle comprising a fragment comprising at least 60% of the full length amino acid sequence of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68.

In one embodiment, the nucleic acid molecule encoding the self-assembling nanoparticle comprises a nucleotide sequence having at least about 90% identity over an entire length of a nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. In one embodiment, the nucleic acid molecule encoding the self-assembling nanoparticle comprises a nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. In one embodiment, the nucleic acid molecule encoding the self-assembling nanoparticle comprises a fragment comprising at least 60% of the full length nucleotide sequence of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67.

In one embodiment, the nucleic acid molecule comprises an expression vector.

In one embodiment, the invention relates to a peptide comprising an amino acid sequence having at least about 90% identity over an entire length of an amino acid sequence of SEQ ID NO:2, SEQ ID NO:4, SEQ ID NO:6 or SEQ ID NO:8. In one embodiment, the peptide comprises SEQ ID NO:2, SEQ ID NO:4, SEQ ID NO:6 or SEQ ID NO: 8. In one embodiment, the invention relates to a polypeptide comprising at least two peptides, wherein each of the at least two peptides comprises an amino acid sequence having at least about 90% identity over an entire length of an amino acid sequence of SEQ ID NO:2, SEQ ID NO:4, SEQ ID NO:6 or SEQ ID NO:8. In one embodiment, the polypeptide comprises at least two of SEQ ID NO:2, SEQ ID NO:4, SEQ ID NO:6 and SEQ ID NO:8.

In one embodiment, the peptide further comprises an oligomerization domain. In one embodiment, the peptide comprises an amino acid sequence having at least about 90% identity over an entire length of an amino acid sequence of SEQ ID NO:12, SEQ ID NO:14, SEQ ID NO:16, SEQ ID NO:18 or SEQ ID NO:20. In one embodiment, the peptide comprises SEQ ID NO:12, SEQ ID NO:14, SEQ ID NO:16, SEQ ID NO:18 or SEQ ID NO:20.

In one embodiment, the invention relates to a self-assembling nanoparticle comprising an amino acid sequence having at least about 90% identity over an entire length of an amino acid sequence selected to SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the amino acid sequence comprises SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In one embodiment, the amino acid sequence comprises a fragment comprising at least 60% of the full length amino acid sequence of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68

In one embodiment, the invention relates to a method of inducing an immune response against SARS Coronavirus 2 (SARS-CoV-2) in a subject in need thereof, the method comprising administering an immunogenic composition comprising a nucleic acid molecule encoding a SARS-CoV-2 spike protein receptor binding domain (RBD), a nucleic acid molecule encoding a SARS-CoV-2 spike protein receptor binding domain (RBD), or a peptide comprising a SARS-CoV-2 spike protein receptor binding domain (RBD) to the subject.

In one embodiment, the method of administering includes at least one of electroporation and injection.

In one embodiment, the invention relates to a method of protecting a subject in need thereof from infection with SARS-CoV-2, the method comprising administering an immunogenic composition comprising a nucleic acid molecule encoding a SARS-CoV-2 spike protein receptor binding domain (RBD), a nucleic acid molecule encoding a SARS-CoV-2 spike protein receptor binding domain (RBD), or a peptide comprising a SARS-CoV-2 spike protein receptor binding domain (RBD) to the subject.

In one embodiment, the method of administering includes at least one of electroporation and injection.

In one embodiment, the invention relates to a method of treating a subject in need thereof against SARS-CoV-2, the method comprising administering an immunogenic composition comprising a nucleic acid molecule encoding a SARS-CoV-2 spike protein receptor binding domain (RBD), a nucleic acid molecule encoding a SARS-CoV-2 spike protein receptor binding domain (RBD), or a peptide comprising a SARS-CoV-2 spike protein receptor binding domain (RBD) to the subject, wherein the subject is thereby resistant to one or more SARS-CoV-2 strains.

In one embodiment, the method of administering includes at least one of electroporation and injection.

### BRIEF DESCRIPTION OF THE DRAWINGS

FIG. 1 depicts data demonstrating that SARS-CoV-2 RBD-Dimers appear more stable structurally.

FIG. 2 depicts the design of nanoparticles to scaffold RBD-dimers for improved immune potency. A single vaccination in BALB/c mice, with 2 ng DNA, was used for each construct.

FIG. 3 depicts data demonstrating the Dl (DNA Launched) nano vaccine (RBD-48mer) elicits rapid neutralizing antibody responses 7 days post immunization.

FIG. 4 depict s the design and testing of additional DNA NanoParticle constructs.

### DETAILED DESCRIPTION OF THE INVENTION

The present invention relates to an immunogenic composition or a vaccine comprising a self-assembling nanoparticle comprising one or more SARS coronavirus 2 (SARS-CoV-2) antigen. SARS-CoV-2 is a highly pathogenic virus, emerging in 2019. The SARS-CoV-2 antigen can be the SARS-CoV-2 spike protein or a fragment thereof. In one embodiment, the SARS-CoV-2 antigen comprises the receptor binding domain (RBD) of the SARS-CoV-2 spike antigen. In one embodiment the immunogenic composition comprises a self-assembling nanoparticle comprising a dimer of the RBD of the SARS-CoV-2 spike antigen.

In one embodiment, the invention relates to a nucleic acid molecule encoding a nanoparticle comprising one or more SARS coronavirus 2 (SARS-CoV-2) antigen. In one embodiment, the nucleic acid molecule encodes a self-assembling nanoparticle comprising the receptor binding domain (RBD) of the SARS-CoV-2 spike antigen. In one embodiment the nucleic acid molecule encodes a self-assembling nanoparticle comprising a dimer of the RBD of the SARS-CoV-2 spike antigen.

The vaccine can be used treat SARS-CoV-2 infection or to prevent or treat a disease or disorder associated with SARS-CoV-2 infection. In one embodiment, the disease or disorder associated with SARS-CoV-2 infection is COVID-19. The vaccine can elicit both humoral and cellular immune responses that target the SARS-CoV-2 spike antigen. The vaccine can elicit neutralizing antibodies and immunoglobulin G (IgG) antibodies that are reactive with the SARS-CoV-2 spike antigen. The vaccine can also elicit CD8⁺ and CD4⁺ T cell responses that are reactive to the SARS-CoV-2 spike antigen and produce interferon-gamma (IFN-γ), tumor necrosis factor alpha (TNF-α), and interleukin-2 (IL-2).

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full length wild type strain SARS-CoV-2 antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a SARS-CoV-2 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Vaccine

Provided herein are immunogenic compositions, such as vaccines, comprising a SARS coronavirus 2 (SARS-CoV-2) antigen, a fragment thereof, a variant thereof, or a combination thereof. The vaccine can be used to treat SARS-CoV-2 infection, thereby treating, preventing, and/or protecting against SARS-CoV-2 based pathologies. In one embodiment, the SARS-CoV-2 based pathology is COVID-19. The vaccine can significantly induce an immune response of a subject administered the vaccine, thereby protecting against and treating SARS-CoV-2 infection.

In one embodiment, the SARS-CoV-2 antigen comprises the receptor binding domain of the SARS-CoV-2 spike protein. In one embodiment, the SARS-CoV-2 antigen comprises a dimer of the receptor binding domain of the SARS-CoV-2 spike protein.

The vaccine can be a DNA vaccine, a peptide vaccine, or a combination DNA and peptide vaccine. The DNA vaccine can include a nucleic acid sequence encoding the SARS-CoV-2 antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the SARS-CoV-2 antigen by a peptide bond. The peptide vaccine can include a SARS-CoV-2 antigenic peptide, a SARS-CoV-2 antigenic protein, a variant thereof, a fragment thereof, or a combination thereof. The combination DNA and peptide vaccine can include the above described nucleic acid sequence encoding the SARS-CoV-2 antigen and the SARS-CoV-2 antigenic peptide or protein, in which the SARS-CoV-2 antigenic peptide or protein and the encoded SARS-CoV-2 antigen have the same amino acid sequence.

In one embodiment, one or more SARS-CoV-2 antigen is incorporated into a self-assembling peptide nanoparticle (SAPN) viral particle for use in a vaccine of the invention. Self-assembling protein nanoparticles (SAPN) may be formed by the assembly of one or more polypeptide chains comprising at least one antigen and at least one protein oligomerization domain. Without limitation, the SAPN of the invention may self-assemble into a tetrahedron, a cube, an octahedron, a dodecahedron, or an icosahedron. The SAPN of the invention may be used as an efficient means for presenting one or more SARS-CoV-2 antigen.

In one embodiment, the SAPN of the invention comprises the receptor binding domain of the SARS-CoV-2 spike protein. In one embodiment, the SAPN of the invention comprises a dimer of the receptor binding domain of the SARS-CoV-2 spike protein.

The vaccine can induce a humoral immune response in the subject administered the vaccine. The induced humoral immune response can be specific for the SARS-CoV-2 antigen. The induced humoral immune response can be reactive with the SARS-CoV-2 antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the SARS-CoV-2 antigen. The neutralizing antibodies can be reactive with the SARS-CoV-2 antigen. The neutralizing antibodies can provide protection against and/or treatment of SARS-CoV-2 infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the SARS-CoV-2 antigen. These IgG antibodies can be reactive with the SARS-CoV-2 antigen. Preferably, the humoral response is cross-reactive against two or more strains of the SARS-CoV-2. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about at least about 15.5-fold, or at least about 16.0-fold as compared to the subject not administered the vaccine.

The vaccine can induce a cellular immune response in the subject administered the vaccine. The induced cellular immune response can be specific for the SARS-CoV-2 antigen. The induced cellular immune response can be reactive to the SARS-CoV-2 antigen. Preferably, the cellular response is cross-reactive against two or more strains of the SARS-CoV-2. The induced cellular immune response can include eliciting a CD8⁺ T cell response. The elicited CD8⁺ T cell response can be reactive with the SARS-CoV-2 antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8+IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the SARS-CoV-2 antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4+IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ+TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly.

### SARS Coronavirus 2 (SARS-CoV-2) Antigen

As described above, in one embodiment, the invention relates to a vaccine comprising a SARS-CoV-2 antigen, a fragment thereof, a variant thereof, or a combination thereof. Coronaviruses, including SARS-CoV-2, are encapsulated by a membrane and have a type 1 membrane glycoprotein known as spike (S) protein, which forms protruding spikes on the surface of the coronavirus. The spike protein facilitates binding of the coronavirus to proteins located on the surface of a cell, for example, the metalloprotease amino peptidase N, and mediates cell-viral membrane fusion. In particular, the spike protein contains an Si subunit that facilitates binding of the coronavirus to cell surface proteins and thus comprises a receptor binding domain (RBD). Accordingly, the Si subunit of the spike protein controls which cells are infected by the coronavirus. In one embodiment, the SARS-CoV-2 antigen of the invention can comprise one or more SARS-CoV-2 spike protein RBD.

The SARS-CoV-2 antigen can be a SARS-CoV-2 spike protein RBD, a fragment thereof, a variant thereof, or a combination thereof. In one embodiment, the composition of the invention comprises a dimer of the SARS-CoV-2 spike protein RBD.

In one embodiment, the composition of the invention is capable of eliciting an immune response in a mammal against one or more SARS-CoV-2 strains. The SARS-CoV-2 antigen can comprise an epitope(s) that makes it particularly effective as an immunogen against which an anti-SARS-CoV-2 immune response can be induced.

The SARS-CoV-2 spike protein RBD can be a consensus sequence derived from two or more strains of SARS-CoV-2. The SARS-CoV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the one or more SARS-CoV-2 spike protein RBD. The one or more SARS-CoV-2 spike protein RBD can comprise a signal peptide such as an immunoglobulin signal peptide, for example, but not limited to, an immunoglobulin E (IgE) or immunoglobulin (IgG) signal peptide.

The SARS-CoV-2 RBD can have an amino acid sequence of SEQ ID NO:2, SEQ ID NO:4, SEQ ID NO:6 or SEQ ID NO:8. In some embodiments, the SARS-CoV-2 RBD can be an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2, SEQ ID NO:4, SEQ ID NO:6 or SEQ ID NO:8.

The nucleic acid molecule encoding the SARS-CoV-2 RBD antigen can comprise the nucleic acid sequence of SEQ ID NO:1, which encodes SEQ ID NO:2. The nucleic acid molecule encoding the SARS-CoV-2 RBD antigen can comprise the nucleic acid sequence of SEQ ID NO:3, which encodes SEQ ID NO:4. The nucleic acid molecule encoding the SARS-CoV-2 RBD antigen can comprise the nucleic acid sequence of SEQ ID NO:5, which encodes SEQ ID NO:6. The nucleic acid molecule encoding the SARS-CoV-2 RBD antigen can comprise the nucleic acid sequence of SEQ ID NO:7, which encodes SEQ ID NO:8. In some embodiments, the nucleic acid molecule encoding the SARS-CoV-2 RBD antigen can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2, SEQ ID NO:4, SEQ ID NO:6 or SEQ ID NO:8. In some embodiments, the nucleic acid molecule encoding the SARS-CoV-2 RBD antigen can comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:1, SEQ ID NO:3, SEQ ID NO:5 or SEQ ID NO:7. In some embodiments, the SARS-CoV-2 RBD antigen can be operably linked to an IgE leader sequence.

Immunogenic fragments of SEQ ID NO:2, SEQ ID NO:4, SEQ ID NO:6 or SEQ ID NO:8 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2 or SEQ ID NO:4. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2, SEQ ID NO:4, SEQ ID NO:6 or SEQ ID NO:8 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% identity to SEQ ID NO:2, SEQ ID NO:4, SEQ ID NO:6 or SEQ ID NO:8. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1, SEQ ID NO:3, SEQ ID NO:5 or SEQ ID NO:7. Immunogenic fragments can be at least 60%, at least 65%, at least 70%, at least 75%, at least 80%, at least 85%, at least 90%, at least 95%, at least 96%, at least 97%, at least 98% or at least 99% of the full length of SEQ ID NO:1, SEQ ID NO:3, SEQ ID NO:5 or SEQ ID NO:7. Immunogenic fragments can comprise at least 95%, at least 96%, at least 97% at least 98% or at least 99% identity to fragments of SEQ ID NO:1, SEQ ID NO:3, SEQ ID NO:5 or SEQ ID NO:7. In some embodiments, immunogenic fragments include sequences that encode a leader sequence, such as for example an immunoglobulin leader, such as the IgE leader. In some embodiments, fragments are free of coding sequences that encode a leader sequence.

SARS-CoV-2 Spike RBD Dimer

The SARS-CoV-2 antigen can be a dimer of the SARS-CoV-2 RBD antigen, a fragment thereof, or a variant thereof. The dimeric SARS-CoV-2 RBD antigen can comprise a linker sequence between the two RBD antigens. The dimeric SARS-CoV-2 RBD antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the outlier SARS-CoV-2 spike antigen. The dimeric SARS-CoV-2 RBD antigen can comprise a signal peptide such as an immunoglobulin signal peptide, for example, but not limited to, an immunoglobulin E (IgE) or immunoglobulin (IgG) signal peptide. The dimeric SARS-CoV-2 RBD antigen can be designed to elicit a stronger cellular and/or humoral immune response than a monomeric SARS-CoV-2 RBD antigen.

The dimeric SARS-CoV-2 RBD antigen can comprise at least two RBD sequences of SEQ ID NO:2, SEQ ID NO:4, SEQ ID NO:6 or SEQ ID NO:8. In one embodiment, the dimeric SARS-CoV-2 RBD antigen comprises two copies of the same SARS-CoV-2 RBD sequence. In one embodiment, the dimeric SARS-CoV-2 RBD antigen comprises two different SARS-CoV-2 RBD sequences. In one embodiment, the dimeric SARS-CoV-2 RBD antigen comprises the amino acid sequence SEQ ID NO:10. In some embodiments, the dimeric SARS-CoV-2 RBD antigen comprises an amino acid sequence having at least about 90%, 91%, 92%, 93%, 94%, 95%, 96%, 97%, 98%, 99%, or 100% identity over an entire length of the amino acid sequence set forth in SEQ ID NO:10.

In some embodiments, the nucleic acid molecule encoding the dimeric SARS-CoV-2 RBD antigen encodes at least two RBD sequences of SEQ ID NO:2, SEQ ID NO:4, SEQ ID NO:6 or SEQ ID NO:8. In some embodiments, the nucleic acid molecule encoding the dimeric SARS-CoV-2 RBD antigen comprises at least of SEQ ID NO:1, SEQ ID NO:3, SEQ ID NO:5 or SEQ ID NO:7. In one embodiment, the nucleic acid molecule encoding the dimeric SARS-CoV-2 RBD antigen encodes two copies of the same SARS-CoV-2 RBD sequence. In one embodiment, the nucleic acid molecule encoding the dimeric SARS-CoV-2 RBD antigen encodes two different SARS-CoV-2 RBD sequences. In one embodiment, the nucleic acid molecule encoding the dimeric SARS-CoV-2 RBD antigen encodes the amino acid sequence SEQ ID NO:10. In one embodiment, the nucleic acid molecule encoding the dimeric SARS-CoV-2 RBD antigen comprises the nucleotide sequence of SEQ ID NO:9. In some embodiments, the nucleic acid molecule encoding the dimeric SARS-CoV-2 RBD antigen comprises a nucleotide sequence that encodes an amino acid sequence having at least about 90%, 91%, 92%, 93%, 94%, 95%, 96%, 97%, 98%, 99%, or 100% identity over an entire length of the amino acid sequence set forth in SEQ ID NO:10. In some embodiments, the nucleic acid molecule encoding the dimeric SARS-CoV-2 RBD antigen comprises a nucleotide sequence having at least about 90%, 91%, 92%, 93%, 94%, 95%, 96%, 97%, 98%, 99% or 100% identity over an entire length of the nucleic acid sequence set forth in SEQ ID NO:9. In some embodiments, the dimeric SARS-CoV-2 RBD antigen can be operably linked to an IgE leader sequence.

Immunogenic fragments of 10 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:10. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:10 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:10. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:9. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:9. Immunogenic fragments can be at least 95%, at least 96%, at least 97% at least 98% or at least 99% homologous to fragments of SEQ ID NO:9. In some embodiments, immunogenic fragments include sequences that encode a leader sequence, such as for example an immunoglobulin leader, such as the IgE leader. In some embodiments, fragments are free of coding sequences that encode a leader sequence.

Self-Assembling NanoParticles

In one embodiment, the invention relates to a vaccine comprising a self-assembling nanoparticle comprising an oligomerization domain and further comprising a SARS-CoV-2 antigen, a fragment thereof, a variant thereof, or a combination thereof. In one embodiment, the invention exploits ferritin, a ubiquitous iron storage protein, that self-assembles into spherical nanoparticles and serves as a scaffold to express a heterologous protein. Therefore in one embodiment, the oligomerization domain comprises ferritin, or a fragment or variant thereof.

In one embodiment, the oligomerization domain comprises a sequence as set forth in SEQ ID NO:12, SEQ ID NO:14, SEQ ID NO:16, SEQ ID NO:18 or SEQ ID NO:20. In some embodiments, the oligomerization domain comprises an amino acid sequence having at least about 90%, 91%, 92%, 93%, 94%, 95%, 96%, 97%, 98%, 99%, or 100% identity over an entire length of the amino acid sequence set forth in SEQ ID NO:12, SEQ ID NO:14, SEQ ID NO:16, SEQ ID NO:18 or SEQ ID NO:20.

In one embodiment, the invention relates to a nucleic acid molecule encoding a self-assembling nanoparticle comprising an oligomerization domain and further comprising a SARS-CoV-2 antigen, a fragment thereof, a variant thereof, or a combination thereof. In some embodiments, the nucleic acid molecule encoding the oligomerization domain can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:12, SEQ ID NO:14, SEQ ID NO:16, SEQ ID NO:18 or SEQ ID NO:20. In some embodiments, the nucleic acid molecule encoding the oligomerization domain can comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:11, SEQ ID NO:13, SEQ ID NO:15, SEQ ID NO:17 or SEQ ID NO:19. In some embodiments, the nucleotide sequence encoding the oligomerization domain can be operably linked to a sequence encoding at least one linker sequence, such as an LS3 or GGS linker sequence.

Immunogenic fragments of SEQ ID NO:12, SEQ ID NO:14, SEQ ID NO:16, SEQ ID NO:18 or SEQ ID NO:20 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:12, SEQ ID NO:14, SEQ ID NO:16, SEQ ID NO:18 or SEQ ID NO:20. Immunogenic fragments of proteins with amino acid sequences homologous to immunogenic fragments of SEQ ID NO:12, SEQ ID NO:14, SEQ ID NO:16, SEQ ID NO:18 or SEQ ID NO:20 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% identity to SEQ ID NO:12, SEQ ID NO:14, SEQ ID NO:16, SEQ ID NO:18 or SEQ ID NO:20.

Some embodiments relate to immunogenic fragments of SEQ ID NO:11, SEQ ID NO:13, SEQ ID NO:15, SEQ ID NO:17 or SEQ ID NO:19. Immunogenic fragments can be at least 60%, at least 65%, at least 70%, at least 75%, at least 80%, at least 85%, at least 90%, at least 95%, at least 96%, at least 97%, at least 98% or at least 99% of the full length of SEQ ID NO:11, SEQ ID NO:13, SEQ ID NO:15, SEQ ID NO:17 or SEQ ID NO:19. Immunogenic fragments can comprise at least 95%, at least 96%, at least 97% at least 98% or at least 99% identity to fragments of SEQ ID NO:11, SEQ ID NO:13, SEQ ID NO:15, SEQ ID NO:17 or SEQ ID NO:19.

In one embodiment, the self-assembling nanoparticle comprising an oligomerization domain and further comprising at least one SARS-CoV-2 RBD domain, referred to herein as the RBD-NP, comprises a sequence as set forth in SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In some embodiments, the RBD-NP comprises an amino acid sequence having at least about 90%, 91%, 92%, 93%, 94%, 95%, 96%, 97%, 98%, 99%, or 100% identity over an entire length of the amino acid sequence set forth in SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68.

In one embodiment, the invention relates to a nucleic acid molecule encoding the RBD-NP, a fragment thereof, a variant thereof, or a combination thereof. In some embodiments, the nucleic acid molecule encoding the RBD-NP can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. In some embodiments, the nucleic acid molecule encoding the RBD-NP can comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. In some embodiments, the nucleotide sequence encoding the oligomerization domain can be operably linked to a sequence encoding at least one linker sequence, such as an LS3 or GGS linker sequence.

Immunogenic fragments of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68. Immunogenic fragments of proteins with amino acid sequences homologous to immunogenic fragments of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% identity SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 or SEQ ID NO:68.

Some embodiments relate to immunogenic fragments of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. Immunogenic fragments can be at least 60%, at least 65%, at least 70%, at least 75%, at least 80%, at least 85%, at least 90%, at least 95%, at least 96%, at least 97%, at least 98% or at least 99% of the full length of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67. Immunogenic fragments can comprise at least 95%, at least 96%, at least 97% at least 98% or at least 99% identity to fragments of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 or SEQ ID NO:67.

Leader Sequence

In some embodiments, the SARS-CoV-2 spike RBD or RBD-NP sequences of the invention are operably linked to at least one leader sequence or a pharmaceutically acceptable salt thereof. In some embodiments, the nucleic acid molecules of the invention encoding the SARS-CoV-2 spike RBD or RBD-NP sequences are operably linked to at least one nucleotide sequence encoding a leader sequence or a pharmaceutically acceptable salt thereof “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

In one embodiment, the leader sequence is the IgE leader sequence comprising the amino acid sequence of MDWTWILFLVAAATRVHS (SEQ ID NO: 69). In some embodiments therefore, the leader sequence in the disclosed expressible nucleic acid sequence comprises a sequence encoding SEQ ID NO:69.

Linker Sequence

In some embodiments, the SARS-CoV-2 spike RBD or RBD-NP sequences of the invention are operably linked to at least one linker sequence. For example, in some embodiments the SARS-CoV-2 spike RBD or RBD-NP sequences comprise a linker between the leader sequence and the SARS-CoV-2 spike RBD sequence. In some embodiments the RBD-NP sequences comprise a linker between the SARS-CoV-2 spike RBD sequence and the oligomerization domain. A linker can be either flexible or rigid or a combination thereof. In one embodiment, the linker is a (GGS)ₙ repeat wherein, the GGS is repeated at least 1, 2, 3, 4, 5, 6, 7, 8, 9, or 10 or more than 10 times.

In some embodiments, the expressible nucleic acid sequence comprises at least one nucleic acid sequence encoding a linker comprising at least 70% sequence identity to SEQ ID NO:70 or a pharmaceutically acceptable salt thereof. SEQ ID NO:70 is the nucleic acid sequence GGAGGCTCCGGAGGATCTGGAGGGAGTGGAGGCTCAGGAGGAGGC encoding the amino acid sequence of GGSGGSGGSGGSGGG (SEQ ID NO: 71). In some embodiments, the at least one nucleic acid sequence, encoding a linker, encodes a sequence comprising at least about 70%, 75%, 80%, 85%, 90%, 95%, or 99% sequence identity to SEQ ID NO:71 or a pharmaceutically acceptable salt thereof. In some embodiments, the at least one nucleic acid sequence, encoding a linker, comprises a sequence having at least about 70%, 75%, 80%, 85%, 90%, 95%, or 99% sequence identity to SEQ ID NO:70 or a pharmaceutically acceptable salt thereof.

In some embodiments, the expressible nucleic acid sequence comprises at least one nucleic acid sequence encoding an LS3 linker comprising at least 70% sequence identity to SEQ ID NO:72 or a pharmaceutically acceptable salt thereof. SEQ ID NO:72 is the nucleic acid sequence TTGCGATTTGGTATTGTCGCTTCCCGCGCAAACCATGCGCTCGTGGGTGGTTCCG GTGGC encoding the amino acid sequence of LRFGIVASRANHALVGGSGG (SEQ ID NO: 73). In some embodiments, the at least one nucleic acid sequence, encoding a linker, encodes a sequence comprising at least about 70%, 75%, 80%, 85%, 90%, 95%, or 99% sequence identity to SEQ ID NO:73 or a pharmaceutically acceptable salt thereof. In some embodiments, the at least one nucleic acid sequence, encoding a linker, comprises a sequence having at least about 70%, 75%, 80%, 85%, 90%, 95%, or 99% sequence identity to SEQ ID NO:72 or a pharmaceutically acceptable salt thereof

Vector

The vaccine can comprise one or more vectors that include a nucleic acid encoding the RBD-NP. The one or more vectors can be capable of expressing the RBD-NP. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

(1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

(2) Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

(3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

Excipients and other Components of the Vaccine

The vaccine may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### Method of Vaccination

Also provided herein is a method of treating, protecting against, and/or preventing disease in a subject in need thereof by administering the vaccine to the subject. Administration of the vaccine to the subject can induce or elicit an immune response in the subject. The induced immune response can be used to treat, prevent, and/or protect against disease, for example, pathologies relating to SARS-CoV-2 infection. In one embodiment, the pathology relating to SARS-CoV-2 infection is COVID-19.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the SARS-CoV-2 spike RBD. The induced cellular immune response can include a CD8⁺ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Feigner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Feigner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### Kit

Provided herein is a kit, which can be used for treating a subject using the method of vaccination described above. In one embodiment, the kit can comprise the vaccine. In one embodiment, the kit can comprise a nucleic acid molecule encoding a RBD-NP of the invention.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, electronic storage media (e.g., magnetic discs, tapes, cartridges), optical media (e.g., CD ROM), and the like. As used herein, the term “instructions” can include the address of an internet site which provides instructions.

The present invention has multiple aspects, illustrated by the following non-limiting examples.

### EXAMPLES

### Example 1

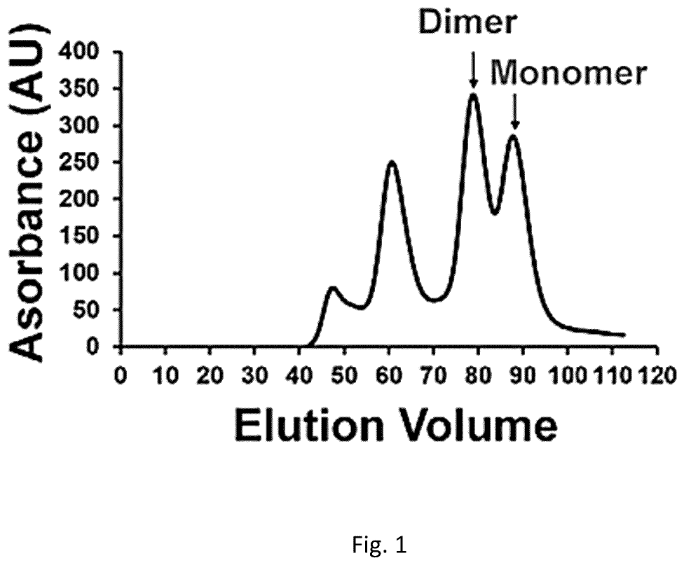

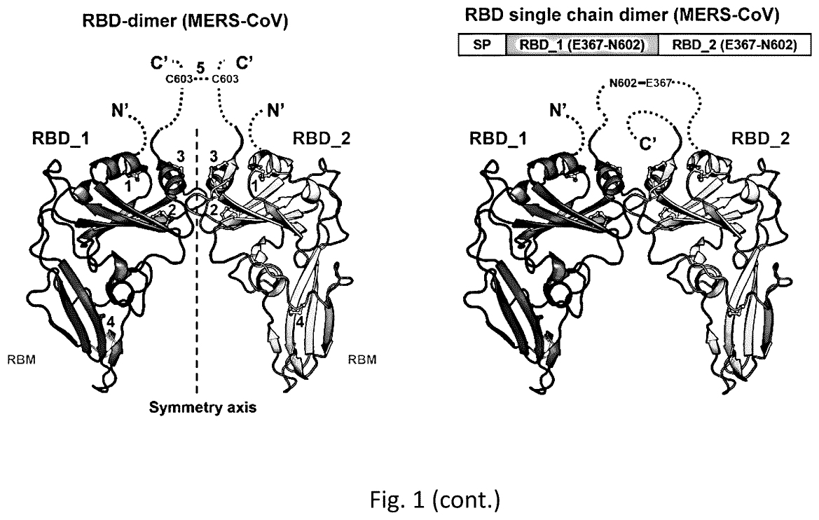

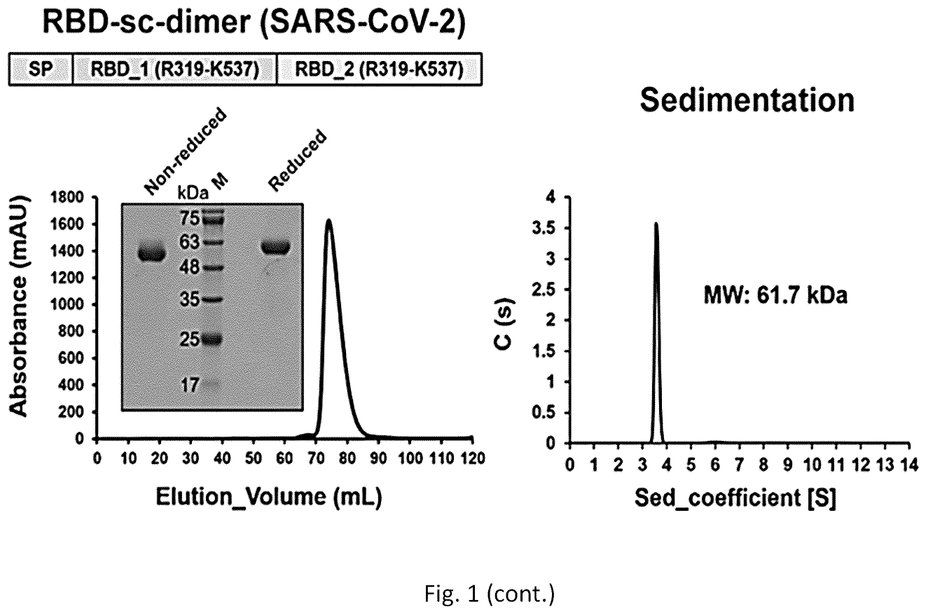

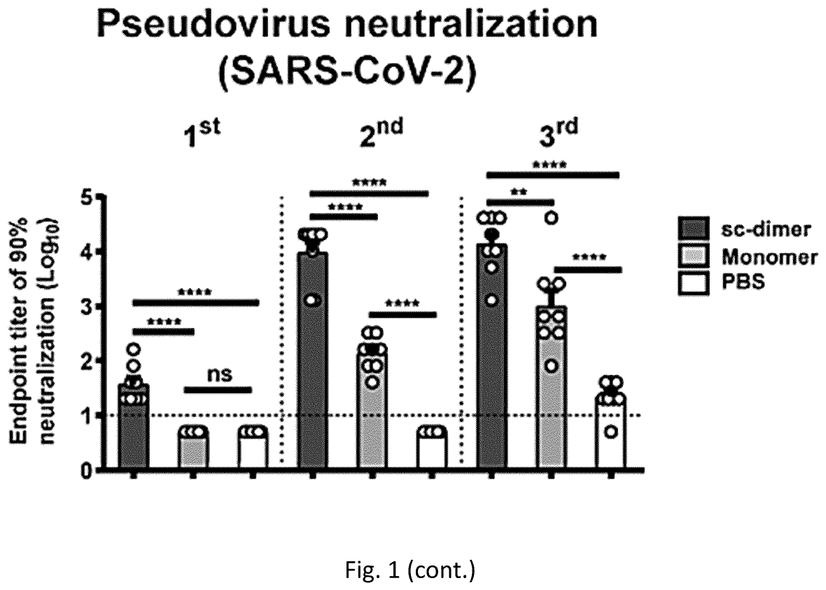

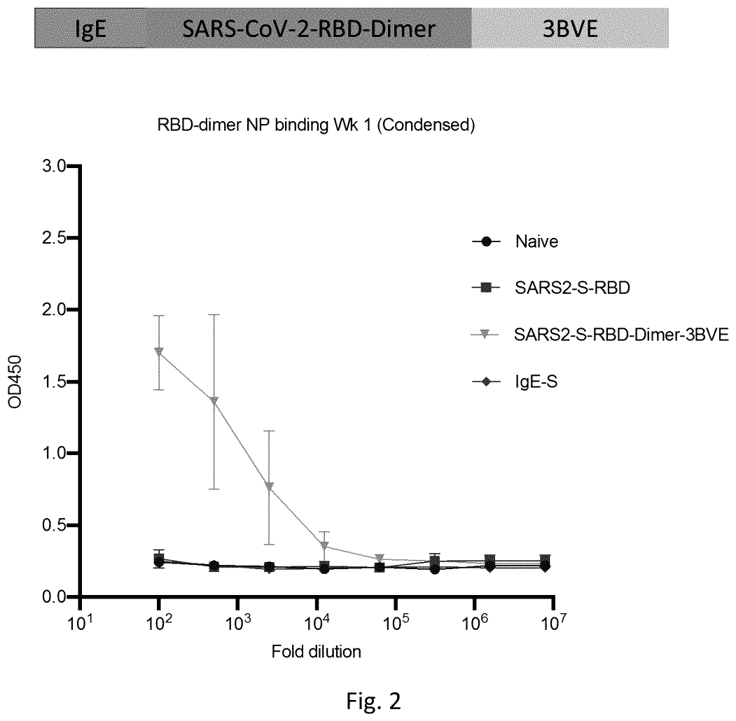

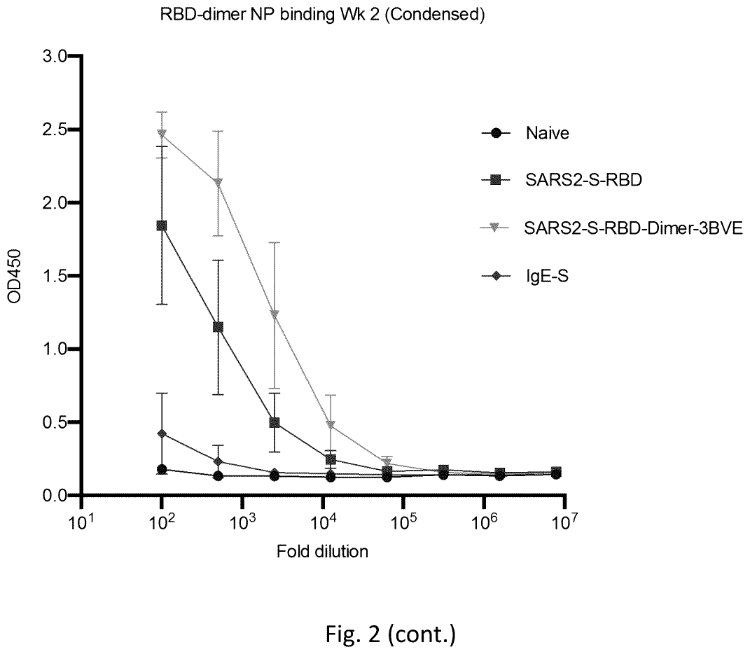

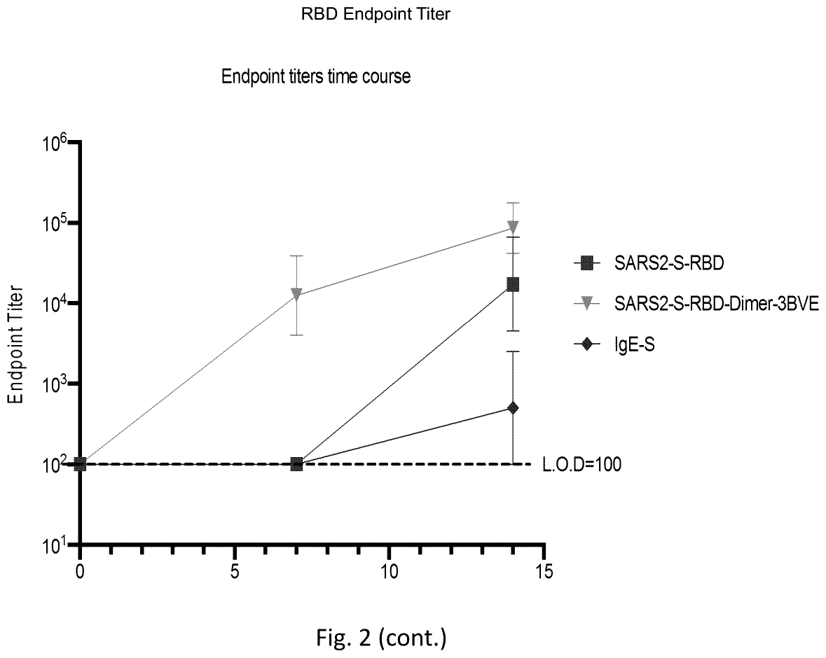

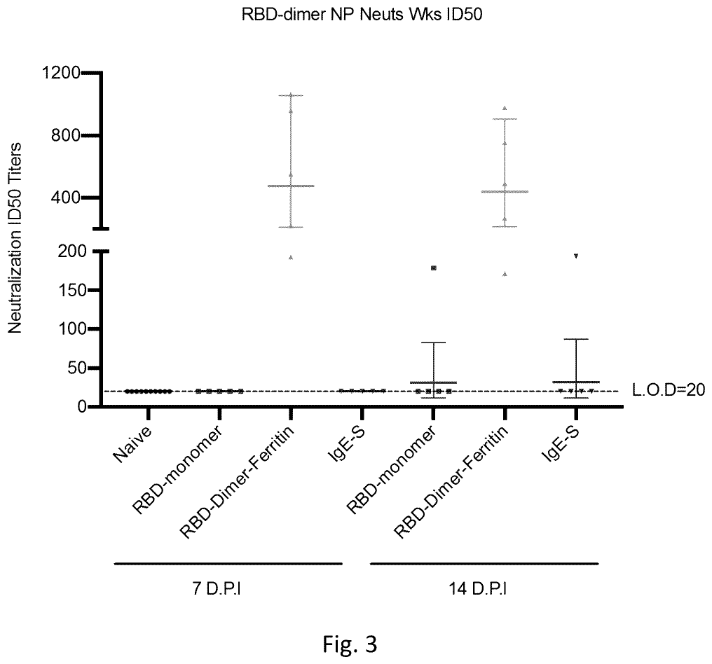

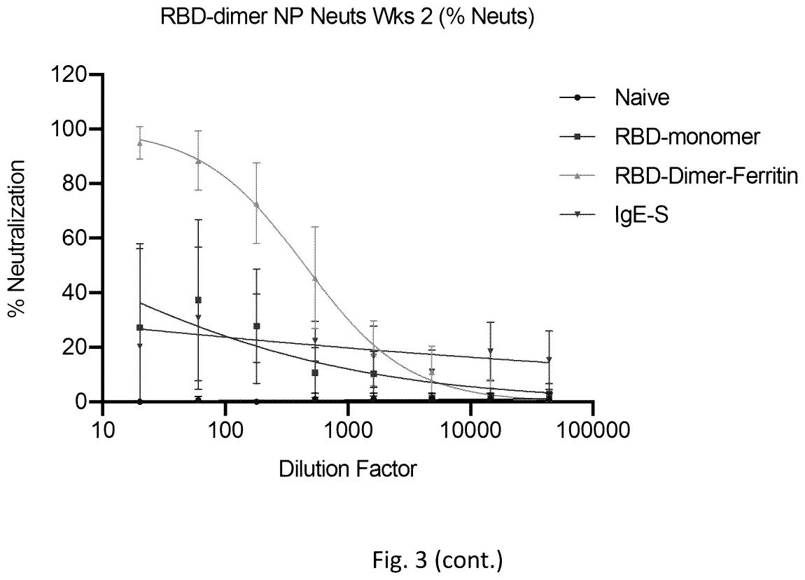

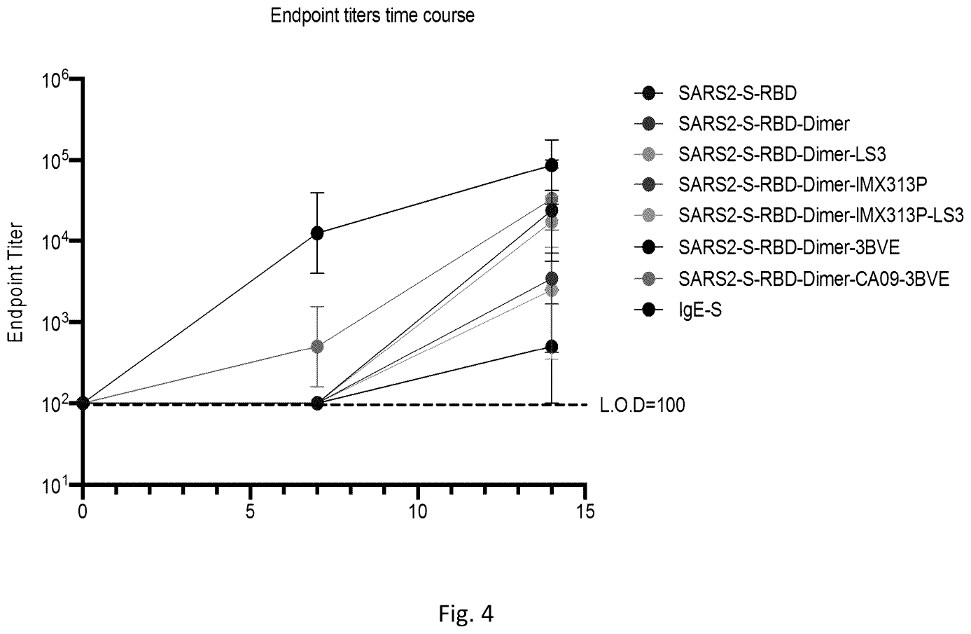

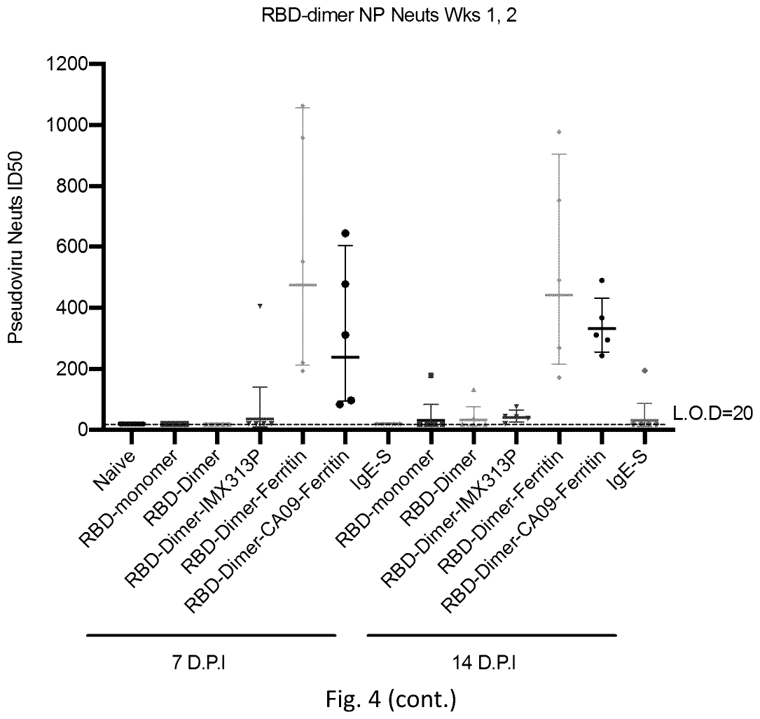

The data presented here demonstrate the development of vaccines for SARS-CoV2 with improved potency. Scaffolds combing RBD binding domains with multivalent self-assembling DNA launched nano particles have been developed (FIG. 1 and FIG. 4). The data demonstrate that the vaccines result in rapid induction of seroconversion by the immunogens, rapid induction of neutralizing titers (FIG. 3) and an overall higher immune potency (FIG. 2).

### Sequences:

| SEQ ID | | |
|---|---|---|
| NO: | Sequence Type | Description |
| 1 | nucleotide | SARS-CoV-2_RBD |
| 2 | amino acid | SARS-CoV-2_RBD |
| 3 | nucleotide | SARS-CoV-2_RBD_2 |
| 4 | amino acid | SARS-CoV-2_RBD_2 |
| 5 | nucleotide | SARS-CoV-2_RBD_3 |
| 6 | amino acid | SARS-CoV-2_RBD_3 |
| 7 | nucleotide | SARS-CoV-2_RBD_4 |
| 8 | amino acid | SARS-CoV-2_RBD_4 |
| 9 | nucleotide | SARS-CoV-2_RBD dimer |
| 10 | amino acid | SARS-CoV-2_RBD dimer |
| 11 | nucleotide | 180 |
| 12 | amino acid | 180 |
| 13 | nucleotide | FR |
| 14 | amino acid | FR |
| 15 | nucleotide | IMX |
| 16 | amino acid | IMX |
| 17 | nucleotide | IMX_2 |
| 18 | amino acid | IMX_2 |
| 19 | nucleotide | LS |
| 20 | amino acid | LS |
| 21 | nucleotide | LS3_RBD_gJohn_180_pVAX |
| 22 | amino acid | LS3_RBD_gJohn_180_pVAX |
| 23 | nucleotide | LS3_RBD_gKylie_180_pVAX |
| 24 | amino acid | LS3_RBD_gKylie_180_pVAX |
| 25 | nucleotide | LS3_RBD_gDan_180_pVAX |
| 26 | amino acid | LS3_RBD_gDan_180_pVAX |
| 27 | nucleotide | LS3_RBD_gPenta_180_pVAX |
| 28 | amino acid | LS3_RBD_gPenta_180_pVAX |
| 29 | nucleotide | LS3_RBD_gJohn_FR_pVAX |
| 30 | amino acid | LS3_RBD_gJohn_FR_pVAX |
| 31 | nucleotide | LS3_RBD_gKylie_FR_pVAX |
| 32 | amino acid | LS3_RBD_gKylie_FR_pVAX |
| 33 | nucleotide | LS3_RBD_gDan_FR_pVAX |
| 34 | amino acid | LS3_RBD_gDan_FR_pVAX |
| 35 | nucleotide | LS3_RBD_gPenta_FR_pVAX |
| 36 | amino acid | LS3_RBD_gPenta_FR_pVAX |
| 37 | nucleotide | LS3_RBD_gJohn_IMX_pVAX |
| 38 | amino acid | LS3_RBD_gJohn_IMX_pVAX |
| 39 | nucleotide | LS3_RBD_gKylie_IMX_pVAX |
| 40 | amino acid | LS3_RBD_gKylie_IMX_pVAX |
| 41 | nucleotide | LS3_RBD_gDan_IMX_pVAX |
| 42 | amino acid | LS3_RBD_gDan_IMX_pVAX |
| 43 | nucleotide | LS3_RBD_gPenta_IMX_pVAX |
| 44 | amino acid | LS3_RBD_gPenta_IMX_pVAX |
| 45 | nucleotide | LS3_RBD_gPenta_IMX_6His_pVAX |
| 46 | amino acid | LS3_RBD_gPenta_IMX_6His_pVAX |
| 47 | nucleotide | RBD_gPenta_dimer_IMX_pVAX |
| 48 | amino acid | RBD_gPenta_dimer_IMX_pVAX |
| 49 | nucleotide | RBDg5_IMX_RBDg5_pVAX |
| 50 | amino acid | RBDg5_IMX_RBDg5_pVAX |
| 51 | nucleotide | RBD_gJohn_LS_pVAX |
| 52 | amino acid | RBD_gJohn_LS_pVAX |
| 53 | nucleotide | RBD_gKylie_LS_pVAX |
| 54 | amino acid | RBD_gKylie_LS_pVAX |
| 55 | nucleotide | RBD_gDan_LS_pVAX |
| 56 | amino acid | RBD_gDan_LS_pVAX |
| 57 | nucleotide | RBD_gPenta_LS_pVAX |
| 58 | amino acid | RBD_gPenta_LS_pVAX |
| 59 | nucleotide | RBD_gPenta_LS_short_pVAX |
| 60 | amino acid | RBD_gPenta_LS_short_pVAX |
| 61 | nucleotide | NTD_gTri4_LS3_RBD_gPenta_pVAX |
| 62 | amino acid | NTD_gTri4_LS3_RBD_gPenta_pVAX |
| 63 | nucleotide | NTD_LS3_RBD_gPenta_pVAX |
| 64 | amino acid | NTD_LS3_RBD_gPenta_pVAX |
| 65 | nucleotide | NTD_gTri4_LS3_RBD_g5_IMX_pVAX |
| 66 | amino acid | NTD_gTri4_LS3_RBD_g5_IMX_pVAX |
| 67 | nucleotide | RBD_g5_dimer_LS_pVAX |
| 68 | amino acid | RBD_g5_dimer_LS_pVAX |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof

What is claimed is:

1. An immunogenic composition comprising a nucleic acid molecule encoding a SARS-CoV-2 spike protein receptor binding domain (RBD), wherein the nucleic acid molecule comprises a nucleotide sequence selected from the group consisting of:
(a) the nucleotide sequence encodes a peptide comprising an amino acid sequence having at least about 90% identity over an entire length of an amino acid sequence selected from the group consisting of SEQ ID NO:2, SEQ ID NO:4, SEQ ID NO:6 and SEQ ID NO:8;
(b) the nucleotide sequence encodes a peptide comprising an amino acid sequence selected from the group consisting of SEQ ID NO:2, SEQ ID NO:4, SEQ ID NO:6 and SEQ ID NO:8;
(c) the nucleotide sequence encodes at least two peptides, wherein each of the at least two peptides comprises an amino acid sequence having at least about 90% identity over an entire length of an amino acid sequence selected from the group consisting of SEQ ID NO:2, SEQ ID NO:4, SEQ ID NO:6 and SEQ ID NO:8; and
(d) the nucleotide sequence encodes at least two peptides, wherein each of the at least two peptides comprises an amino acid sequence selected from the group consisting of SEQ ID NO:2, SEQ ID NO:4, SEQ ID NO:6 and SEQ ID NO:8.

2. The immunogenic composition of claim 1, wherein the nucleic acid molecule comprises a nucleotide sequence selected from the group consisting of:
(a) the nucleotide sequence having at least about 90% identity over an entire length of the nucleic acid sequence selected from the group consisting of SEQ ID NO:1, SEQ ID NO:3, SEQ ID NO:5 and SEQ ID NO:7;
(b) the nucleic acid sequence selected from the group consisting of SEQ ID NO:1, SEQ ID NO:3, SEQ ID NO:5 and SEQ ID NO:7;
(c) the nucleotide sequence encodes at least two peptides, wherein each of the encoding sequences comprises at least about 90% identity over an entire length of a nucleotide sequence selected from the group consisting of SEQ ID NO:1, SEQ ID NO:3, SEQ ID NO:5 and SEQ ID NO:7; and
(d) the nucleotide sequence encodes at least two peptides, wherein each of the encoding sequences comprises a nucleotide sequence selected from the group consisting of SEQ ID NO:1, SEQ ID NO:3, SEQ ID NO:5 and SEQ ID NO:7.

3. The immunogenic composition of claim 1, wherein the nucleic acid molecule further encodes an oligomerization domain selected from the group consisting of:
(a) a peptide comprising an amino acid sequence having at least about 90% identity over an entire length of an amino acid sequence selected from the group consisting of SEQ ID NO:12, SEQ ID NO:14, SEQ ID NO:16, SEQ ID NO:18 and SEQ ID NO:20; and
(b) a peptide comprising an amino acid sequence selected from the group consisting of SEQ ID NO:12, SEQ ID NO:14, SEQ ID NO:16, SEQ ID NO:18 and SEQ ID NO:20.

4. The immunogenic composition of claim 3, wherein the nucleotide sequence encoding the oligomerization domain is selected from the group consisting of:
(a) a nucleotide sequence having at least about 90% identity over an entire length of a nucleotide sequence selected from the group consisting of SEQ ID NO:11, SEQ ID NO:13, SEQ ID NO:15, SEQ ID NO:17 and SEQ ID NO:19; and
(b) a nucleotide sequence selected from the group consisting of SEQ ID NO:11, SEQ ID NO:13, SEQ ID NO:15, SEQ ID NO:17 and SEQ ID NO:19.

5. The immunogenic composition of claim 1, wherein the nucleic acid molecule encodes self-assembling nanoparticle comprising an amino acid sequence selected from the group consisting of:
(a) an amino acid sequence having at least about 90% identity over an entire length of an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68;
(b) an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68; and
(c) a fragment comprising at least 60% of the full length amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68.

6. The immunogenic composition of claim 5, wherein the nucleic acid molecule encoding the self-assembling nanoparticle comprises a nucleotide sequence selected from the group consisting of:
(a) a nucleotide sequence having at least about 90% identity over an entire length of a nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67;
(b) a nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67; and
(c) a fragment comprising at least 60% of the full length nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67.

7. The immunogenic composition of claim 1, wherein the nucleic acid molecule comprises an expression vector.

8. The immunogenic composition of claim 1, wherein the nucleic acid molecule is incorporated into a viral particle.

9. The immunogenic composition of claim 1, further comprising a pharmaceutically acceptable excipient.

10. The immunogenic composition of claim 1, further comprising an adjuvant.

11. A nucleic acid molecule comprising a nucleotide sequence encoding a SARS-CoV-2 spike protein receptor binding domain (RBD), wherein the nucleic acid molecule comprises a nucleotide sequence selected from the group consisting of:
(a) the nucleotide sequence encodes a peptide comprising an amino acid sequence having at least about 90% identity over an entire length of an amino acid sequence selected from the group consisting of SEQ ID NO:2, SEQ ID NO:4, SEQ ID NO:6 and SEQ ID NO:8;
(b) the nucleotide sequence encodes a peptide comprising an amino acid sequence selected from the group consisting of SEQ ID NO:2, SEQ ID NO:4, SEQ ID NO:6 and SEQ ID NO:8;
(c) the nucleotide sequence encodes at least two peptides, wherein each of the at least two peptides comprises an amino acid sequence having at least about 90% identity over an entire length of an amino acid sequence selected from the group consisting of SEQ ID NO:2, SEQ ID NO:4, SEQ ID NO:6 and SEQ ID NO:8; and
(d) the nucleotide sequence encodes at least two peptides, wherein each of the at least two peptides comprises an amino acid sequence selected from the group consisting of SEQ ID NO:2, SEQ ID NO:4, SEQ ID NO:6 and SEQ ID NO:8.

12. The nucleic acid molecule of claim 11, wherein the nucleic acid molecule comprises a nucleotide sequence selected from the group consisting of:
(a) the nucleotide sequence having at least about 90% identity over an entire length of the nucleic acid sequence selected from the group consisting of SEQ ID NO:1, SEQ ID NO:3, SEQ ID NO:5 and SEQ ID NO:7;
(b) the nucleic acid sequence selected from the group consisting of SEQ ID NO:1, SEQ ID NO:3, SEQ ID NO:5 and SEQ ID NO:7;
(c) the nucleotide sequence encodes at least two peptides, wherein each of the encoding sequences comprises at least about 90% identity over an entire length of a nucleotide sequence selected from the group consisting of SEQ ID NO:1, SEQ ID NO:3, SEQ ID NO:5 and SEQ ID NO:7; and
(d) the nucleotide sequence encodes at least two peptides, wherein each of the encoding sequences comprises a nucleotide sequence selected from the group consisting of SEQ ID NO:1, SEQ ID NO:3, SEQ ID NO:5 and SEQ ID NO:7.

13. The nucleic acid molecule of claim 11, wherein the nucleic acid molecule further encodes an oligomerization domain selected from the group consisting of:
(a) a peptide comprising an amino acid sequence having at least about 90% identity over an entire length of an amino acid sequence selected from the group consisting of SEQ ID NO:12, SEQ ID NO:14, SEQ ID NO:16, SEQ ID NO:18 and SEQ ID NO:20; and
(b) a peptide comprising an amino acid sequence selected from the group consisting of SEQ ID NO:12, SEQ ID NO:14, SEQ ID NO:16, SEQ ID NO:18 and SEQ ID NO:20.

14. The nucleic acid molecule of claim 13, wherein the nucleotide sequence encoding the oligomerization domain is selected from the group consisting of:
(a) a nucleotide sequence having at least about 90% identity over an entire length of a nucleotide sequence selected from the group consisting of SEQ ID NO:11, SEQ ID NO:13, SEQ ID NO:15, SEQ ID NO:17 and SEQ ID NO:19; and
(b) a nucleotide sequence selected from the group consisting of SEQ ID NO:11, SEQ ID NO:13, SEQ ID NO:15, SEQ ID NO:17 and SEQ ID NO:19.

15. The nucleic acid molecule of claim 11, wherein the nucleotide sequence encodes self-assembling nanoparticle comprising an amino acid sequence selected from the group consisting of:
(a) an amino acid sequence having at least about 90% identity over an entire length of an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68;
(b) an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68; and
(c) a fragment comprising at least 60% of the full length amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68.

16. The nucleic acid molecule of claim 15, wherein the nucleic acid molecule encoding the self-assembling nanoparticle comprises a nucleotide sequence selected from the group consisting of:
(a) a nucleotide sequence having at least about 90% identity over an entire length of a nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67;
(b) a nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67; and
(c) a fragment comprising at least 60% of the full length nucleotide sequence selected from the group consisting of SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65 and SEQ ID NO:67.

17. The nucleic acid molecule of claim 11, wherein the nucleic acid molecule comprises an expression vector.

18. A peptide comprising an amino acid sequence selected from the group consisting of:
(a) a peptide comprising an amino acid sequence having at least about 90% identity over an entire length of an amino acid sequence selected from the group consisting of SEQ ID NO:2, SEQ ID NO:4, SEQ ID NO:6 and SEQ ID NO:8;
(b) a peptide comprising an amino acid sequence selected from the group consisting of SEQ ID NO:2, SEQ ID NO:4, SEQ ID NO:6 and SEQ ID NO:8;
(c) an amino acid sequence comprising at least two peptides, wherein each of the at least two peptides comprises an amino acid sequence having at least about 90% identity over an entire length of an amino acid sequence selected from the group consisting of SEQ ID NO:2, SEQ ID NO:4, SEQ ID NO:6 and SEQ ID NO:8; and
(d) an amino acid sequence comprising at least two peptides, wherein each of the at least two peptides comprises an amino acid sequence selected from the group consisting of SEQ ID NO:2, SEQ ID NO:4, SEQ ID NO:6 and SEQ ID NO:8.

19. The peptide of claim 18 further comprising an oligomerization domain selected from the group consisting of:
(a) a peptide comprising an amino acid sequence having at least about 90% identity over an entire length of an amino acid sequence selected from the group consisting of SEQ ID NO:12, SEQ ID NO:14, SEQ ID NO:16, SEQ ID NO:18 and SEQ ID NO:20; and
(b) a peptide comprising an amino acid sequence selected from the group consisting of SEQ ID NO:12, SEQ ID NO:14, SEQ ID NO:16, SEQ ID NO:18 and SEQ ID NO:20.

20. The peptide of claim 18 comprising a self-assembling nanoparticle comprising an amino acid sequence selected from the group consisting of:
(a) an amino acid sequence having at least about 90% identity over an entire length of an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68;
(b) an amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68; and
(c) a fragment comprising at least 60% of the full length amino acid sequence selected from the group consisting of SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66 and SEQ ID NO:68

21. A method of inducing an immune response against SARS Coronavirus 2 (SARS-CoV-2) in a subject in need thereof, the method comprising administering an immunogenic composition of claim 1, a nucleic acid molecule of claim 11 or a peptide of claim 18 to the subject.

22. The method of claim 21, wherein administering includes at least one of electroporation and injection.

23. A method of protecting a subject in need thereof from infection with SARS-CoV-2, the method comprising administering an immunogenic composition of claim 1, a nucleic acid molecule of claim 11 or a peptide of claim 18 to the subject.

24. The method of the claim 23, wherein administering includes at least one of electroporation and injection.

25. A method of treating a subject in need thereof against SARS-CoV-2, the method comprising administering an immunogenic composition of claim 1, a nucleic acid molecule of claim 11 or a peptide of claim 18 to the subject, wherein the subject is thereby resistant to one or more SARS-CoV-2 strains.

26. The method of claim 25, wherein administering includes at least one of electroporation and injection.
